(19) United States
(12) Patent Application Publication
(10) Pub. No.: US 2026/0183134 A1
(43) Pub. Date: Jul. 2, 2026

(54) INSERTABLE ANTI-LEAKING STOMA OR OSTOMY DEVICE

(71) Applicant: Paige Cisna, Granger, IN (US)

(72) Inventor: Paige Cisna, Granger, IN (US)

(21) Appl. No.: 19/550,031

(22) Filed: Feb. 25, 2026

Related U.S. Application Data
(63) Continuation of application No. 18/665,409, filed on May 15, 2024.

(51) Int. Cl. A61F 5/445

(52) U.S. Cl. CPC A61F 5/445

(57) ABSTRACT

An insertable anti-leaking ostomy device includes a tube having an internal end and an external end, and a first inflatable balloon positioned at or near the internal end. The device is configured to be inserted through a stoma into a patient's body, with the first inflatable balloon positioned inside the body and the external end remaining outside. After insertion, the first inflatable balloon is inflated via an inflation valve to retain the device beneath a fascial layer and to provide a sealed flow channel for effluent. In some embodiments, a second inflatable balloon is positioned at or near the external end to help funnel effluent into a normally attached ostomy pouch and reduce leakage. An applicator with a base and a plunger can facilitate insertion and deployment of the device.

### CROSS-REFERENCE TO RELATED APPLICATIONS

This application is a continuation of U.S. patent application Ser. No. 18/665,409, filed on May 15, 2024, and claims the benefit of that application under 35 U.S.C. § 120. The entire disclosure of the above-referenced application is hereby incorporated herein by reference.

### STATEMENT REGARDING FEDERALLY SPONSORED RESEARCH OR DEVELOPMENT

Not applicable.

### REFERENCE TO A SEQUENCE LISTING, A TABLE, OR A COMPUTER PROGRAM LISTING APPENDIX

Not applicable.

### FIELD

The present disclosure relates to ostomy management devices and, more particularly, to insertable stoma or ostomy devices that reduce peristomal leakage by providing a sealed flow channel for effluent.

### BACKGROUND

An ostomy is a surgically created opening that permits discharge of bodily waste through a stoma, for example, in connection with a colostomy, ileostomy, or urostomy. Ostomy patients frequently rely on external adhesive barriers and collection pouches to manage effluent.

A persistent challenge is leakage around the stoma. Leakage is particularly problematic for ileostomies and urostomies that produce liquid effluent, and it can cause skin irritation, breakdown, infection risk, increased supply usage, and reduced quality of life. Conventional solutions primarily target external sealing, and may be less effective for challenging anatomies such as retracted stomas, uneven peristomal contours, or widened fascial openings (e.g., due to parastomal hernia).

Accordingly, there remains a need for ostomy devices and methods that reduce leakage by establishing a secure, controlled effluent pathway from within the body to a collection pouch.

### SUMMARY

The present disclosure provides an insertable anti-leaking stoma or ostomy device. In various embodiments, the device includes a tube defining a lumen and having an internal end and an external end. A first inflatable balloon is positioned at or near the internal end and is configured to be deployed within the body cavity connected to the stoma, for example, beneath a fascial layer. When inflated, the first inflatable balloon can inhibit withdrawal of the tube through the fascial opening and can help maintain patency of the lumen for effluent flow.

In various embodiments, an inflation assembly accessible from outside the body is fluidly coupled to the first inflatable balloon via an inflation passage (e.g., an inflation lumen formed in a wall of the tube). The inflation assembly can include an inflation port and/or valve configured to receive a syringe or other fluid source for delivering inflation fluid (e.g., sterile water or saline).

In some embodiments, the device includes a second inflatable balloon positioned at or near the external end of the tube. The second inflatable balloon, when inflated, can facilitate use with an ostomy pouch and/or can help funnel effluent into the ostomy pouch to further reduce leakage. The second inflatable balloon may be inflated via the same inflation assembly used for the first inflatable balloon, or via a separate inflation assembly.

The present disclosure also provides methods of inserting and deploying the device using an applicator. In various embodiments, the method includes inserting at least the internal end of the tube and a deflated first inflatable balloon through the stoma using the applicator, deploying the device such that the first inflatable balloon is positioned beneath the fascial layer, and inflating one or more balloons to retain the tube and direct effluent through the lumen to an ostomy pouch.

### BRIEF DESCRIPTION OF THE DRAWINGS

The accompanying drawings illustrate embodiments of the present disclosure and, together with the description, explain principles of the disclosure. Like reference numerals generally refer to like elements throughout the drawings.

FIG. 1 is a cross-sectional view of an embodiment of an insertable anti-leaking ostomy device after insertion through a stoma, illustrating a first inflatable balloon positioned beneath a fascial layer.

FIG. 2 is a cross-sectional view of an alternative embodiment of an insertable anti-leaking ostomy device including a first inflatable balloon and a second inflatable balloon, with an ostomy pouch in place.

FIG. 3 is a perspective view of an applicator and plunger, with the insertable anti-leaking ostomy device positioned within the applicator prior to insertion.

### DETAILED DESCRIPTION

The following description is provided to enable a person of ordinary skill in the art to make and use embodiments of the present disclosure. Various modifications will be apparent to those skilled in the art, and the general principles described herein may be applied to other embodiments. Accordingly, the present disclosure is not intended to be limited to the specific embodiments described, but is to be accorded the widest scope consistent with the principles and features disclosed.

### Definitions

As used herein, the terms “internal” and “external” are used with reference to the patient when the device is installed. The internal end of the tube is the end positioned within the body cavity within the bowel lumen, and the external end of the tube is the end positioned outside the body for coupling with a collection pouch (e.g., an ostomy pouch that is normally adhered to the patient's peristomal skin in the usual fashion). As used herein, the term “fascial layer” refers to a fascial plane of the abdominal wall (e.g., an anterior rectus sheath and/or other abdominal wall fascia) that defines a fascial opening through which the stoma tract extends. The terms “proximal” and “distal” may also be used with the same orientation, unless otherwise stated.

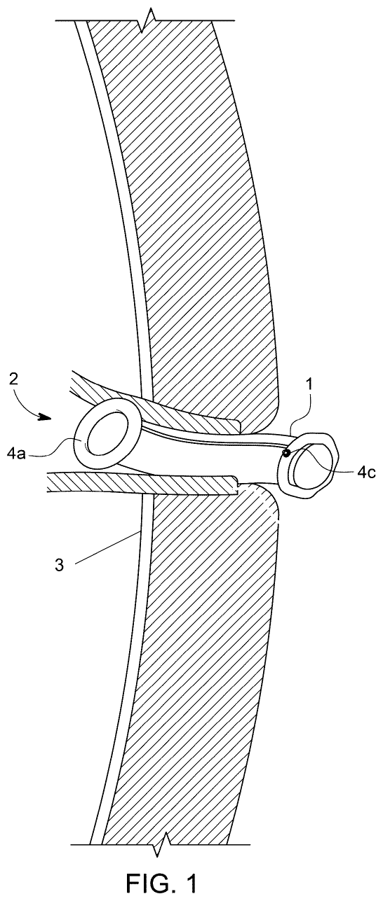

Referring to FIG. 1, an insertable anti-leaking ostomy device 1 includes a tube 2 defining a lumen configured to convey effluent. The tube 2 extends through a stoma tract and through a fascial layer 3. The tube 2 includes an internal portion configured to be positioned within the body cavity and an external portion configured to remain outside the body.

A first inflatable balloon 4a is positioned at or near the internal portion of the tube 2. In various embodiments, the first inflatable balloon 4a is annular (ring-shaped) and defines a central opening aligned with the lumen of the tube 2 to permit effluent flow through the lumen while the balloon is inflated. When inflated, the first inflatable balloon 4a can expand to a diameter larger than the fascial opening, thereby retaining the tube 2 beneath the fascial layer 3 and inhibiting inadvertent withdrawal through the stoma.

The device 1 can further include an inflation assembly 4c accessible from outside the body. The inflation assembly 4c is fluidly coupled to the first inflatable balloon 4a via an inflation passage (e.g., an inflation lumen extending within a wall of the tube 2). The inflation assembly 4c can include a valve, port, septum, or other connector configured to receive inflation fluid from a syringe or other source to inflate the first inflatable balloon 4a with sterile water, saline, or other medically acceptable inflation medium.

In some embodiments, the inflation assembly 4c can be integrated with, or located adjacent to, an external collar, cuff, flange, or other external feature that assists with positioning the device relative to a conventional ostomy appliance (e.g., an adhesive skin barrier and a collection pouch). In such embodiments, the ostomy pouch is typically adhered to the patient's peristomal skin in the usual fashion, and the external portion of the tube 2 extends through (or is received within) an opening of the ostomy appliance such that effluent discharged from the lumen is directed into the ostomy pouch. In embodiments that omit a second inflatable balloon (e.g., FIG. 1), the external collar or other external feature can help provide a stable interface for use with the ostomy appliance.

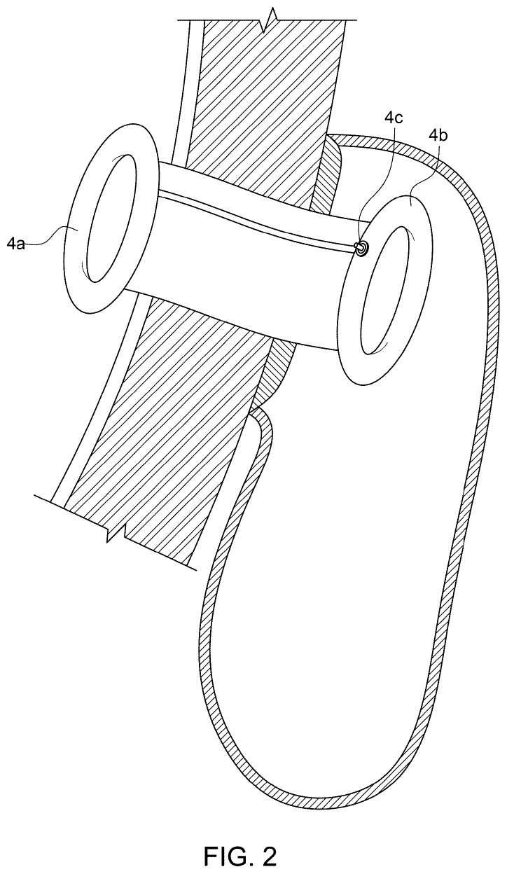

Referring to FIG. 2, in some embodiments the device 1 further includes a second inflatable balloon 4b positioned at or near the external portion of the tube 2. The second inflatable balloon 4b may be annular (ring-shaped). When inflated, the second inflatable balloon 4b can help funnel effluent into an ostomy pouch and/or can improve sealing of the ostomy pouch by reducing risk of leaking at the pouch interface.

In various embodiments, the second inflatable balloon 4b may be inflated through the inflation assembly 4c (e.g., via a shared inflation passage network within the wall of the tube 2). In other embodiments, the second inflatable balloon 4b is fluidly coupled to a separate second inflation assembly (not shown) that is distinct from the inflation assembly 4c used for the first inflatable balloon 4a. Where separate inflation assemblies are provided, the assemblies may be differentiated by color, shape, tactile features, labeling, or other indicia to reduce confusion during use.

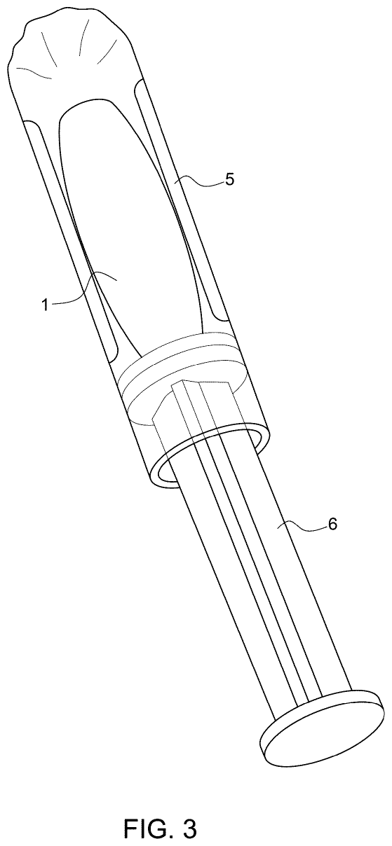

Referring to FIG. 3, the device 1 may be provided for insertion using an applicator 5 and a plunger 6. In one example method, the internal portion of the tube 2 and the deflated first inflatable balloon 4a are rolled or folded and loaded into the applicator 5. The applicator 5 is inserted through the stoma until a distal tip of the applicator 5 is positioned beyond the fascial layer 3. The plunger 6 is advanced to deploy the device 1 from the applicator 5, after which the applicator 5 is withdrawn while leaving the device 1 in place.

After deployment, one or more balloons (e.g., first inflatable balloon 4a and, where present, second inflatable balloon 4b) are inflated by delivering inflation fluid through the inflation assembly 4c and/or a separate inflation assembly. The ostomy pouch can be coupled with the device before or after balloon inflation, depending on clinician preference and the particular embodiment and/or pouch type.

The tube 2 and inflatable balloon(s) may be formed from flexible, biocompatible materials suitable for extended contact with bodily tissue and fluids, including, by way of example, silicone or other elastomeric materials. The tube 2 can be configured to resist kinking while maintaining lumen patency. In various embodiments, the device is provided in multiple sizes (e.g., different tube lengths and/or balloon diameters) to accommodate patient anatomy such as thickness of subcutaneous tissue, degree of stoma retraction, and/or widened fascial openings.

In some use cases, the device 1 may be periodically replaced. For removal, inflation fluid may be withdrawn through the inflation assembly 4c (and/or any separate inflation assembly) to deflate the inflatable balloon(s), after which the device 1 is withdrawn through the stoma.

While specific embodiments have been described, various changes and modifications may be made without departing from the spirit and scope of the disclosure. Unless the context clearly requires otherwise, the singular terms “a”, “an”, and “the” include plural referents, and the term “comprising” is intended to be open-ended.

What is claimed is:

1. An insertable anti-leaking ostomy device, comprising:
a tube having a first end and a second end;
a first inflatable balloon positioned at the first end of the tube; and
a first balloon valve fluidly connected to the first inflatable balloon,
wherein the first end and the first inflatable balloon are configured to be inserted through a stoma into a patient's body cavity.

2. The insertable anti-leaking ostomy device of claim 1, wherein the first inflatable balloon is ring-shaped, forming a hollow center aligned with a lumen of the tube.

3. The insertable anti-leaking ostomy device of claim 1, wherein the first inflatable balloon is configured to be positioned beneath a fascial layer inside the patient's body when inflated.

4. The insertable anti-leaking ostomy device of claim 1, wherein the first balloon valve is accessible from outside the patient's body when the device is inserted.

5. The insertable anti-leaking ostomy device of claim 1, further comprising:
a second inflatable balloon positioned at the second end of the tube; and
a second balloon valve fluidly connected to the second inflatable balloon.

6. The insertable anti-leaking ostomy device of claim 5, wherein the second inflatable balloon is ring-shaped, forming a hollow center aligned with a lumen of the tube.

7. The insertable anti-leaking ostomy device of claim 5, wherein the second inflatable balloon is configured to help funnel effluent into an ostomy pouch when inflated.

8. The insertable anti-leaking ostomy device of claim 1, wherein the tube is made of flexible, biocompatible material.

9. The insertable anti-leaking ostomy device of claim 8, wherein the tube is made of silicone.

10. The insertable anti-leaking ostomy device of claim 1, wherein the device is configured to be used with an applicator for insertion through the stoma.

11. The insertable anti-leaking ostomy device of claim 10, wherein the applicator comprises:
a plastic base with a soft silicone tip; and
a plunger configured to deploy the device from the plastic base.

12. The insertable anti-leaking ostomy device of claim 1, wherein the first inflatable balloon is configured to be inflated with sterile water or saline solution.

13. The insertable anti-leaking ostomy device of claim 1, wherein the tube is available in different lengths to accommodate varying patient anatomies.

14. The insertable anti-leaking ostomy device of claim 1, wherein the device is configured to extend a retracted stoma beyond abdominal contours to direct effluent into an ostomy pouch.

15. A method of inserting an anti-leaking ostomy device, comprising:
rolling a first end of a tube with a deflated first balloon;
inserting the rolled first end into an applicator;
inserting the applicator through a stoma;
deploying the first end and the deflated first balloon from the applicator into a body cavity connected to the stoma;
removing the applicator while leaving the first end and the deflated first balloon in the body cavity; and
inflating the first balloon via a balloon valve to secure the device in position.

16. The method of claim 15, further comprising:
attaching an ostomy pouch to the patient's skin around a second end of the tube that remains outside the patient's body.

17. The method of claim 15, wherein the first balloon is inflated with sterile water or saline solution.

18. The method of claim 15, further comprising:
inflating a second balloon positioned at a second end of the tube that remains outside the patient's body.

19. The method of claim 15, wherein deploying the first end and the deflated first balloon comprises pushing a plunger of the applicator to push the first end and the deflated first balloon out of the applicator.

20. The method of claim 15, further comprising: selecting a tube length based on a thickness of subcutaneous fat of the patient.
